(19) United States
(12) Patent Application Publication
(10) Pub. No.: US 2002/0082412 A1
(43) Pub. Date: Jun. 27, 2002

(54) Methods

(75) Inventors: John Charles Brennand, Macclesfield (GB); Kevin Anthony Hart, Macclesfield (GB)

Correspondence Address: ROTHWELL, FIGG, ERNST & MANBECK, P.C., 555 13TH STREET, N.W., SUITE 701, EAST TOWER, WASHINGTON, DC, 20004, US

(73) Assignee: AstraZeneca AB, Sodertalje (SE)

(21) Appl. No.: 09/845,720

(22) Filed: May 2, 2001

Related U.S. Application Data
(60) Provisional application No. 60/201,417, filed on May 3, 2000.

(51) Int. Cl. A01K 67/00; C07H 21/04; A61K 38/17; C07K 14/705

(52) U.S. Cl. 536/23.5; 514/12; 800/8; 530/350

(57) ABSTRACT

The use of the G protein coupled receptor GPR3 to identify appetite control agents and diagnostic agents.

This invention relates to the regulation of metabolism and in particular to human genes involved in appetite control or obesity. The invention also relates to the identification of ligands that interact with receptors encoded by such genes and the provision of therapeutic agents.

Obesity is now a major health problem. Currently 22.5% of the US population is considered to be clinically obese, 18.5% in the UK and with many other developed countries following this trend. It has been described as the most extensive non-communicable disease of the 21st century. Currently available treatments are reviewed by M. Lean in Exp. Clin. Endocrinol. Diabetes, 1998, 106, Suppl. 2, 22-26. These include diet and, in extreme cases, surgery.

It is only in recent years that the genetic basis and influences on obesity have been studied in detail. By some estimates 40-70% of the variation in obesity-related phenotypes in humans is heritable. The search for human obesity genes is conveniently summarised by Comuzzie et al (Science, 1998, 280, 1374-1377). In particular, leptin (LEP), the product of the ob gene, and the leptin receptor (LEPR) have now been studied in detail. Leptin is a hormone secreted by adipose tissue which, along with its receptor, are integral parts of the complex physiological system which has evolved to regulate and control energy balance and storage at an optimal level (Freidman J M and Halaas J L 1998 Nature 395, 763-769). Leptin also appears to play an important role in relaying nutritional status to several other physiological systems. The relevance of leptin to the pathogenesis of obesity in general is the subject of much study and underlines the complex nature of human obesity. A human obesity gene map is now available and the number of genes and other markers that have been associated or linked with human obesity phenotypes now approaches 200.

A large number of products are being developed for the treatment of obesity and eating disorders and these are targeted against a wide range of biological targets. The chosen targets include enzymes, hormones, neurotransmitters as well as the so-called G-protein-coupled-receptors (GPCRs). The GPCRs represent one of the largest families of genes so far identified. Over 800 family members have been cloned to date from a wide variety of species.

Our investigations have now revealed that the mRNA coding for a G-protein-coupled-receptor, GPR3, is differentially expressed in rodent appetite/obesity models. It follows that peptidic and non-peptidic compounds which modulate the biological activity of GPR3, will have utility in controlling food intake and metabolic processes.

GPR3 is a receptor with no known physiological ligand, i.e. it is an “Orphan Receptor”. It has greatest amino acid identity to members of sub-class of receptors (GPR6 & GPR12) which are themselves “Orphan” (Iismaa ., 1994, Genomics, 15 391-394; Song et al., 1995, Genomics, 28, 347-349). GPR3 has also 26% amino acid identity to the melanocortin family of receptors. The human gene is located on chromosome lp34.3 (ibid). Expression in the peripheral tissue is low and is found in lung and kidney. It is also expressed within the brain.

cDNA encoding human GPR3 has been cloned by Song Z H et al.,1995, Genomics, 28, 347-349; Iismaa T P et al., 1994, Genomics, 24, 391-394; Eggerickx D et al., 1995, Biochem. J. 309, 837-843 and Marchese A et al., 1994, Genomics, 23 609-618). A partial cDNA has been published in the Genbank database under accession number g577416. GPR3 amino acid sequence is published in the EMBL database under accession number P46089. A murine homologue of GPR3 has been cloned by Saeki Y et al.,1993, FEBS Lett 336 317-322. The amino acid sequence of the murine homologue is published in the EMBL database under accession number P35413.

Therefore in a first aspect of the present invention we provide a method for the provision of an appetite control agent which method comprises using one or more agonists and/or antagonists of the G protein coupled receptor GPR3 as test compounds in one or more appetite control test procedures, and selecting an active compound for use as an appetite control agent.

Convenient appetite control test procedures include the use of animal models to test the role of the test compound in appetite control and obesity. These will typically involve the administration of compounds by intra peritoneal injection, subcutaneous injection, intravenous injection, oral gavage or direct injection via canullae into the CNS of experimental animals. The effects on food intake, body temperature, metabolic rate, behavioural activities and body weight changes may all be measured using standard procedures.

Suitable antagonists or agonists may be firstly identified by screening for agonists and/or antagonists of GPR3.

Therefore in a further aspect of the present invention we provide a method for the provision of an appetite control agent which method comprises (i) screening for agonists and/or antagonists of GPR3 and (ii) using one or more agonists and/or antagonists so identified as test compounds in one or more appetite control test procedures, and selecting an active compound for use as an appetite control agent.

GPR3 is from any mammalian species, including human, rat, mouse, monkey, and dog. For screening purposes GPR3 is conveniently human GPR3.

Mammalian GPR3 may be conveniently isolated from commercially available RNA, brain cDNA libraries, genomic DNA, or genomic DNA libraries using conventional molecular biology techniques such as library screening and/or polymerase chain reaction (PCR). These techniques are extensively detailed in Molecular Cloning—A Laboratory Manual, 2^{nd} edition, Sambrook, Fritsch & Maniatis, Cold Spring Harbor Press.

The resulting cDNA's encoding mammalian GPR3 are then cloned into commercially available mammalian expression vectors such as pcDNAIII (InVitrogen Ltd etc. see below). An alternative mammalian expression vector is disclosed by Davies et al., J of Pharmacol & Toxicol. Methods, 33, 153-158. Standard transfection technologies are used to introduce these DNA's into commonly available cultured, mammalian cell lines such as CHO, HEK293, HeLa and clonal derivatives expressing the receptors are isolated. An alternative expression system is the MEL cell expression system claimed in our UK patent no. 2251622.

Application of a natural ligand to these cells causes activation of the transfected receptor that will cause changes in the levels of intracellular signalling molecules such as cyclic-AMP, intracellular calcium ions or arachidonic acid metabolite release. These may all be measured using standard published procedures and commercially available reagents. In addition, the receptor cDNA's may be transfected into derivatives of these cells lines that have previously been transfected with a “reporter” gene such as bacterial LacZ, Luciferase, aequorin or green fluorescent protein that will “report” these intracellular changes.

The natural ligand for GPR3 is not yet known. The cells transfected with GPR3 may be used to find natural ligands that will activate GPR3. Ligands may be sourced commercially or synthesised chemically (Lembo et al., 1999, Nature Cell Biol., 1, 267-271) or may be purified from mammalian sources such as animal brain extracts (Saurai et al., 1998, Cell, 92, 573-585). Once identified, purified, radiolabelled or fluorescently labelled material (eg. Amersham PLC & Advanced Bioconcept Ltd) may be used as a ligand to detect ligand binding to the transfected receptors using standard published ligand binding assay technologies.

The transfected cell lines may be used to identify low molecular weight compounds that activate the receptors and cause changes in intracellular signalling molecules which mimic the effects of the natural ligand, these are defined as “agonists”.

In addition or alternatively, the same assays can be used to identify low molecular weight compounds that prevent activation of the receptors and suppress the effects of the natural ligand, these are defined as “antagonists”.

The test compound may be a polypeptide of equal to or greater than 2 amino acids, such as up to 6 amino acids, up to 10 or 12 amino acids, up to 20 amino acids or greater than 20 amino acids such as up to 50 amino acids. For drug screening purposes, preferred compounds are chemical compounds of low molecular weight and potential therapeutic agents. They are for example of less than about 2000 daltons, such as less than 1500, 1000, 800, 600 or 400 daltons in weight. If desired the test compound may be a member of a chemical library. This may comprise any convenient number of individual members, for example tens to hundreds to thousands to millions of suitable compounds, for example peptides, peptoids and other oligomeric compounds (cyclic or linear), and template-based smaller molecules, for example benzodiazepines, hydantoins, biaryls, carbocyclic and polycyclic compounds (eg. naphthalenes, phenothiazines, acridines, steroids etc.), carbohydrate and amino acids derivatives, dihydropyridines, benzhydryls and heterocycles (eg. triazines, indoles, thiazolidines etc.). The numbers quoted and the types of compounds listed are illustrative, but not limiting. Preferred chemical libraries comprise chemical compounds of low molecular weight and potential therapeutic agents.

In a further aspect of the invention we provide the use of an agonist of GPR3 as an appetite control agent.

In a further aspect of the invention we provide the use of an antagonist of GPR3 as an appetite control agent.

It will be appreciated that the present invention includes the use of orthologues and homologues of human GPR3.

By the term “orthologue” we mean the functionally equivalent receptor in other species.

By the term “homologue” we mean a substantially similar and/or related receptor in the same or a different species.

For either of the above definitions we believe the receptors may have for example at least 30%, such as at least 40%, at least 50%, at least 60%, and in particular at least 70%, such as at least 80%, for example 85%, or 90% or 95% peptide sequence identity. It is appreciated that homologous receptors may have substantially higher peptide sequence identity over small regions representing functional domains. We include receptors having greater diversity in their DNA coding sequences than outlined for the above amino acid sequences but which give rise to receptors having peptide sequence identity falling within the above sequence ranges. Convenient versions of GPR3 include the published sequence (ref al.ibid) and the sequence identities set out in Tables 1 to 3.

Fragments and partial sequences of the GPR3 may be useful substrates in the assay and analytical methods of the invention. It will be appreciated that the only limitation on these is practical, they must comprise the necessary functional elements for use in the relevant assay and/or analytical procedures.

In a further aspect of the present invention we provide a method of appetite control which method comprises administering to an individual a pharmaceutically effective amount of an appetite control agent identified using one or more of the methods of this invention.

The appetite control agent of this invention may be administered in standard manner for the condition that it is desired to treat, for example by oral, topical, parenteral, buccal, nasal, or rectal administration or by inhalation. For these purposes the compounds of this invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions.

Knowledge of the GPR3 gene provides the ability to regulate its expression in vivo by for example the use of antisense oligonucleotides. Thus, according to a further aspect of the invention we provide an appetite control agent comprising an antisense oligonucleotide which is complementary to all or a part of a polynucleotide sequence shown in Table 1. By complementary we mean that the two molecules can hybridise to form a double stranded molecule through nucleotide base pair interactions. U.S. Pat. No. 5,639,595, Identification of novel Drugs and Reagents, issued Jun. 17, 1997, wherein methods of identifying oligonucleotide sequences that display in vivo activity are thoroughly described, is herein incorporated by reference.

The antisense oligonucleotide for co-operation with a polynucleotide sequence corresponding to all or a part of a GPR3 gene may be produced using conventional means, by standard molecular biology and/or by chemical synthesis. If desired, the antisense oligonucleotide may be chemically modified so as to prevent degradation in vivo or to facilitate passage through a cell membrane and/or a substance capable of inactivating mRNA, for example ribozyme, may be linked thereto. Examples of antisense molecules include but are not limited to DNA, stable derivatives of DNA such as phosphorothioates or methylphosphonates, RNA, stable derivatives of RNA such as 2′-O-alkylRNA, or other oligonucleotide mimetics such as peptide nucleic acids. U.S. Pat. No. 5,652,355, Hybrid Oligonucleotide Phosphorothioates, issued Jul. 29, 1997, and U.S. Pat. No. 5,652,356, Inverted Chimeric and Hybrid Oligonucleotides, issued Jul. 29, 1997, which describe the synthesis and effect of physiologically-stable antisense molecules, are herein incorporated by reference.

The antisense oligonucleotide can be complementary to the full length GPR3 gene of the invention or to a fragment thereof. Antisense molecules which comprise oligomers in the range from about 12 to about 30 nucleotides which are complementary to the regions of the gene which are proximal to, or include, the protein coding region, or a portion thereof, are preferred embodiments of the invention. GPR3 gene antisense molecules may be introduced into cells by microinjection, liposome encapsulation or by expression from vectors harboring the antisense sequence.

GPR3 may also be used as the basis for diagnosis, for example to determine expression levels in a human subject, by for example direct DNA sequence comparison or DNA/RNA hybridisation assays. Diagnostic assays may involve the use of nucleic acid amplification technology such as PCR and in particular the Amplification Refractory Mutation System (ARMS) as claimed in our European Patent No. 0 332 435. Such assays may be used to determine allelic variants of the gene, for example insertions, deletions and/or mutations such as one or more point mutations. Such variants may be heterozygous or homozygous. Other approaches have been used to identify mutations in genes encoding similar molecules in obese patients (Yeo et al., 1998, Nature Genetics, 20, 111-112).

In a further aspect of the invention GPR3 can be genetically engineered in such a way that its interactions with other intracellular and membrane associated proteins are maintained but its effector function and biological activity are removed. The genetically modified protein is known as a dominant negative mutant. Overexpression of the dominant negative mutant in an appropriate cell type down regulates the effect of the endogenous protein, thus revealing the biological role of the genes in appetite control.

Similarly, GPR3 may also be genetically engineered in such a way that its effector function and biological activity are enhanced. The resultant overactive protein is known as dominant positive mutant. Overexpression of a dominant positive mutant in an appropriate cell type amplifies the biological response of the endogenous, native protein, spotlighting its role in appetite control. This also has utility in a screen for detecting antagonists of the constitutively active receptor in the absence of a ligand.

Therefore, in a further aspect of the invention we provide dominant negative and dominant positive mutants of GPR3 and their use in evaluating the biological role of GPR3 in the control of appetite.

Transgenic animal technology is also contemplated, providing new experimental models, useful for evaluating the effects of test compounds on the control of obesity and eating disorders. GPR3 genes may be deleted, inactivated or modified using standard procedures as outlined briefly below and as described for example in “Gene Targeting; A Practical Approach”, IRL Press, 1993. The target gene or a portion of it, for example homologous sequences flanking the coding region, is preferably cloned into a vector with a selection marker (such as Neo) inserted into the gene to disrupt its function. The vector is linearised then transformed (usually by electroporation) into embryonic stem cells (ES) cells (eg derived from a 129/Ola strain of mouse) and thereafter homologous recombination events take place in a proportion of the stem cells. The stem cells containing the gene disruption are expanded and injected into a blastocyst (such as for example from a C57BL/6J mouse) and implanted into a foster mother for development. Chimaeric offspring may be identified by coat colour markers. Chimaeras are bred to ascertain the contribution of the ES cells to the germ line by mating to mice with genetic markers which allow a distinction to be made between ES derived and host blastocyst derived gametes. Half of the ES cell derived gametes will carry the gene modification. Offspring are screened (for example by Southern blotting) to identify those with a gene disruption (about 50% of the progeny). These selected offspring will be heterozygous and may therefore be bred with another heterozygote to produce homozygous offspring (about 25% of the progeny).

Transgenic animals with a target gene deletion (“knockouts”) may be crossed with transgenic animals produced by known techniques such as microinjection of DNA into pronuclei, sphaeroplast fusion or lipid mediated transfection of ES cells to yield transgenic animals with an endogenous gene knockout and a foreign gene replacement. ES cells containing a targeted gene disruption may be further modified by transforming with the target gene sequence containing a specific alteration. Following homologous recombination the altered gene is introduced into the genome. These embryonic stem cells may subsequently be used to create transgenics as described above.

The transgenic animals will display a phenotype which reflects the role of GPR3 in the control of appetite and obesity and will thus provide useful experimental models in which to evaluate the effects of test compounds. Therefore in a further aspect of the invention we provide transgenic animals in which GPR3 genes are deleted, inactivated or modified, and their use in evaluating the effects of test compounds in appetite control and obesity.

The invention will now be illustrated but not limited by reference to the following specific description and sequence tables [Many of the specific techniques used are detailed in standard molecular biology textbooks such as Sambrook, Fritsch & Maniatis, Molecular cloning, a Laboratory Manual, Second Edition, 1989, Cold Spring Harbor Laboratory Press. Consequently references to this will be made at the appropriate points in the text.]:

PCR Cloning of GPR3

Oligonucleotide primers of 30 nucleotides in length corresponding to sequences immediately 5′ of the initiating ATG codon and immediately 3′ of the termination codon for the coding sequences of human and rodent GPR3 (sequences below) are synthesised. Commercial sources of rodent and human brain RNA are used as templates in standard RT-PCR reactions with these primers. RT-PCR primers are designed to incorporate nucleotides coding for tag sequences e.g. myc, His 6 to facilitate purification of the proteins at a later stage. Commercially available RT-PCR kits are used in accordance with the suppliers instructions and as documented in the Sambrook reference cited above. Products of the PCR vector are cloned using standard technology (ibid) into the plasmid vector pBluescript (Stratagene Ltd.). Plasmid DNA is isolated (ibid) and subjected to DNA sequence analysis (ibid) to identify a clone containing the GPR3 sequence identical to those listed below. The inserts corresponding to GPR3 cDNA are released from this DNA using standard digestion procedures and with appropriate restriction endonuclease enzymes. The inserts are then cloned into suitably prepared plasmid DNA using standard technology (ibid). These plasmids are the expression vectors used in the studies described below.

Cloning Into Expression Vectors

(i) A variety of mammalian expression vectors may be used to express the recombinant GPR3 molecule as well as variants contemplated herein. Commercially available mammalian expression vectors which are suitable for recombinant expression, include but are not limited to, pcDNA3 (Invitrogen), pMC1neo (Stratagene), pXT1 (Stratagene), pSG5 (Stratagene), EBO-pSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and 1ZD35 (ATCC 37565), pLXIN and pSIR (Clontech), pIRES-EGFP (Clontech). Plasmid DNA containing the GPR3 cDNA inserts is then purified (ibid) and introduced into appropriate host cells.

(ii) A vector is described for use with the Mouse Erythroleukaemia Cells (MEL) expression system using the human beta globin gene locus control region (Davies et al., J of Pharmacol & Toxicol. Methods, 33, 153-158.). This vector system and derivatives thereof may also be used. Plasmid DNA containing the GPR3 cDNA inserts is then purified (ibid) and introduced into appropriate host cells.

Transfection/Selection of Host Cells

(i) Mammalian expression vector plasmid DNA is introduced (ibid) into cultured mammalian cells. Eukaryotic recombinant host cells are especially preferred. Examples include but are not limited to yeast, mammalian cells including but not limited to cell lines of human, bovine, porcine, monkey and rodent origin, and insect cells including but not limited to Drosophila and silkworm derived cell lines. Cell lines derived from mammalian species which may be suitable and which are commercially available, include but are not limited to, L cells L-M(TK-) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1(ATCC CCL 61), 3T3 (ATCC CCL 92), NIH/3T3 (ATCC CRL 1658), HeLa (ATCC CCL 2), C1271 (ATCC CRL 1616),BS-C-1 (ATCC CCL 26), MRC-5 (ATCC CCL 171) and HEK293 (ATCC CRL 1573). In addition the DNA is introduced into variants of these cell lines that have previously been transfected and selected to express other proteins such as β-galactosidase, or mutated G-proteins such as Ga16 (Milligan et al, 1996, TiPS, 17, 235-237). Clones of mammalian cells expressing GPR3 cDNA are identified by selecting mammalian cell clones that have been selected on the basis of their resistance to antbiotics due to the presence of appropriate resistance genes on the parental plasmids (See Maniatis, et al), by RT-PCR of the introduced sequences and by detection of protein using specific antibodies.

(ii) The DNA containing the beta-globin locus control region and GPR3 cDNA is introduced into MEL cells, clones are selected and analysed as described (Davies et al. op cit).

The expression vectors may be introduced into host cells expressing GPR3 via any one of a number of techniques including but not limited to transformation, transfection, lipofection, protoplast fusion, and electroporation. Commercially available kits applicable for use with the present invention for hererologous expression, including well-characterised vectors, transfection reagents and conditions, and cell culture materials are well-established and readily available. [CLONTECH, Palo Alto, Calif.; INVITROGEN, Carlsbad, Calif.; PHARMINGEN, San Diego, Calif.; STRATAGENE, LaJolla, Calif.]

Identification of Ligands for GPR3.

Identification of the natural ligand for GPR3 entails successive purification and assay steps using rat, porcine, or other animal brain as starting material. Homogenised brain tissue is fractionated by conventional biochemical methods and fractions are screened for activity in the reporter cell assays described below. Detailed protocols for these methods are available (Sakurai, et al. 1998, Cell, 92:573-585). Successive purification procedures yield a purified ligand for GPR3 that is characterised by sequencing methodologies (ibid).

Cell Binding Assay

Mammalian cells isolated from the selection procedures described above are cultured by standard techniques and exposed to ¹²⁵[I] ligand. Following extensive washing of cells to remove unbound material the extent of ligand binding is quantitated in a Gammamaster counter (Packard) using the methods described in detail by Davies et al. (op cit). Cell clones showing the greatest binding of this ligand are progressed to the next phase of this process.

Membrane Preparation

The mammalian cell clones identified by the method described above are cultured, harvested and used as the source of membrane preparation. Membranes are prepared from these cell clones by standard biochemical techniques that are described in detail by Davies et al. (op cit).

Ligand Binding Assays

(i) Cell membranes isolated from these mammalian cell clones are used to establish conventional ligand binding assays as described in detail in Davies et al. (op cit). or:

(ii) The same membranes are used with the same radioligand or with GTPg[S]35 to develop a scintillation proximity assay (SPA) using proprietary SPA beads developed by Amersham Ltd. Licences and detailed protocols for this technique are available from Amersham Ltd.

Reporter Cell Assays :- cAMP/Ca++flux/arachidonic acid metabolite release

Cells expressing GPR3 are identified as described above. These cells have also been engineered to express the LacZ gene coupled to the mammalian cyclic AMP response element (Egerton et al, J.Mol.Endocrinol, 1995, 14(2), 179-189). When cAMP levels increase in the cell the transcription of the LacZ gene is proportionately increased and may be measured by standard beta-galactosidase assays (Maniatis et al., ibid).

Cells expressing GPR3 are also engineered to express the G-protein Ga16 (Milligan et al., 1996, TiPS, 17, 235-237). Upon activation the cells respond by increasing intracellular calcium concentrations. This increase is measured after pre-exposure of the cells to a fluorescent compound such as, but not limited to, Fura2 (Molecular Probes Ltd) by reading on any commercially available fluorescence analysing equipment (Lembo et al., 1999, Nature Cell Biol., 1, 267-271).

Cells expressing GPR3 are also assayed for the increased release of radiolabelled arachidonic acid metabolites following pre-incubation of the cells to ³[H] arachidonic acid and stimulation by PrRP31 (Davies et al., ibid).

Compound Screening

Chemical compounds are tested for their ability to inhibit (antagonise) the biological activity of GPR3 and to increase (agonise) the activity of GPR3.

(i) The ligand binding and SPA assays described above are conducted in the presence of varying amounts of individual compounds that will reveal those compounds that have the ability to displace the natural ligands from GPR3.

(ii) These compounds are applied to the mammalian cells in the presence and absence of ligand and those compounds that influence the output of the assays described above are identified.

The following methods, which do not require the presence of any ligand, are also suitable for identifying chemical compounds having the desired properties. Agonists: The reporter cells containing GPR3 are exposed to chemical compounds in the absence of any ligand, and assayed, as described, for changes in intracellular cAMP, and Ca++ as well as for increased arachidonic acid metabolite release. Antagonists: The GPR3 cDNA is mutated using standard molecular biology techniques (Maniatis, ibid) and transfected into the mammalian reporter cells, as described. Cell lines harbouring mutated receptors that give increased reporter gene activity are then used to screen chemical compounds for their ability to suppress this reporter gene activity through antagonising the constitutively active receptors.

Compound Testing In Vivo

Compounds identified from the assays described above are considered for testing in animal models. Appropriately formulated compounds are administered by, but not limited to, oral gavage, intraperitoneal, intravenous, intramuscular or intracerebrovascular injection or infusion. Animals will include, but are not limited to, standard laboratory rodents, dogs and primates, obese Zucker rats, obese (ob/ob) mice, and diabetic (db/db) mice. The animals may be fed standard laboratory diets, or may be offered altered diets, including but not limited to, diets designed to induce hyperphagia and weight gain, for example high fat, high carbohydrate (Stock, 1998, Clinical Obesity, Oxford Press, 50-72). The effect of compound on the following, but not limited to, will be established: food intake parameters, water intake, body weight changes, body fat, protein and water composition, endocrine parameters, metabolic substrate concentrations, energy expenditure and behavioural activities, using standard physiological, biochemical and neurobiological methods (Halford et al, 1998, Pharmacol. Biochem. Behav., 61, 159-168, Shimada et al, 1998, Nature, 396, 670-674).

Antibody Production

The GPR3 polypeptide can be used to raise diagnostic antibodies to detect the receptor in cultured cells and in vivo. Therefore, in accordance with yet a further aspect of the present invention, there are provided antibodies against the GPR3 polypeptide which may be used as part of various diagnostic assays for detecting physiological eating disorders. An example for the production of effective polyclonal antibodies against peptides derived from the known amino acid sequences of GPR3 utilises a well established algorithm method developed by Jameson and Wolf, The antigenic Index: A novel Algorithm for Predicting Antigenic Determinants, CABIOS, 4:181 (1988). Peptide molecules of typically between 10-20 amino acid residues are synthesised chemically and conjugated to keyhole limpet hemocyanin and used for antibody generation by Genosys Biotechnologies, 1442 Lake Front Circle, Suite 185, The Woodlands, Tex. 77380. Specific antibodies may be raised by immunising animals, with rabbits being preferred, with an appropriate concentration of the GPR3 peptides either with or without an immune adjuvant.

Monospecific antibodies to the polypeptide of the present invention are purified from mammalian antisera containing antibodies reactive against the GPR3 polypeptide using the technique of Kohler and Milstein, Nature, 256:495 (1975). Mono-specific antibody as used herein is defined as a single antibody species or multiple antibody species with homogenous binding characteristics for the novel signal transduction molecule. Homogenous binding as used herein refers to the ability of the antibody species to bind to a specific antigen or epitope, such as those associated with the sequences set out in tables 2 and 3. Monoclonal antibodies are produced in vivo by injection of pristane primed Balb/c mice, approximately 0.5 ml per mouse, with about 2×10⁶ to about 6×10⁶ hybridoma cells about 4 days after priming. Ascites fluid is collected at approximately 8-12 days after cell transfer and the monoclonal antibodies are purified by techniques known in the art. In vitro production of the anti-polypeptide mAb is carried out by growing the hydridoma in DMEM containing about 2% foetal calf serum to obtain sufficient quantities of the specific mAb. The mAb are purified by techniques known in the art.

TABLE 1
Human GPR3 Partial cDNA
Genbank accession number g577416
cccaggaagagacccctgtttagaggcctgggggcattggaggggacagcggtatcctgggaagagccccagggcatgaatgtgg
ggataaggcattgggaccctatcaggtatcctgaggagagactcccaccacgtatcctgagaagcacctcaccccctccagaccccaa
ctcccatcacccagcttggtcagcttctcacaaggcctttctcctgcaggtaccatgatgtggggtgcaggcagccctctggcctggctct
cagctggctcaggcaacgtgaatgtaagcagcgtgggcccagcagaggggcccacaggtccagccgcaccactgccctcgcctag
gcctgggatgtggtgctctgcatctcaggcaccctggtgtcctgcgagaatgcgctagtggtggccatcatcgtgggcactcctgccttc
cgtgcccccatgttcctgctggtgggcagcctggccgtggcagacctgctggcaggcctgggcctggtcctgcactttgctgctgtcttc
tgcatcggctcagcggagatgagcctggtgctggttggcgtgctggcaatggcctttaccgccagcatcggcagtctactggccatcac
tgtcgaccgctacctttctctgtacaatgccctcacctactattcagagacaacagtgacacggacctatgtgatgctggccttagtgtggg
gaggtgccctgggcctggggctgctgcctgtgctggcctggaactgcctggatggcctgaccacatgtggcgtggtttatccactctcc
aagaaccatctggtagttctggccattgccttcttcatggtgtttggcatcatgctgcagctctacgcccaaatctgccgcatcgtctgccg
ccatgcccagcagattgcccttcagcggcacctgctgcctgcctcccactatgtggccacccgcaagggcattgccacactggccgtg
gtgcttggagcctttgccgcctgctggttgcccttcactgtctactgcctgctgggtgatgcccactctccacctctacacctatcttacct
tgctccctgccacctacaactccatgatc

TABLE 2
Human GPR3 amino acid sequence
EMBL accession number P46089
MMWGAGSPLAWLSAGSGNVNVSSVGPAEGPTGPAAPLPSPKAWDVVLCISG
TLVSCENALVVAIIVGTPAFRAPMFLLVGSLAVADLLAGLGLVLHFAAVFC
IGSAEMSLVLVGVLAMAFTASIGSLLAITVDRYLSLYNALTYYSETTVTRT
YVMLALVWGGALGLGLLPVLAWNCLDGLTTCGVVYPLSKNHLVVLAIAFFM
VFGIMLQLYAQICRIVCRHAQQIALQRHLLPASHYVATRKGIATLAVVLGA
FAACWLPFTVYCLLGDAHSPPLYTYLTLLPATYNSMINPIIYAFRNQDVQK
VLWAVCCCCSSSKIPFRSRSPSDV

TABLE 3
Murine homologue of GPR3
EMBL accession number P35413
MMWGAGSSMAWFSAGSGSVNVSSVDPVEEPTGPATLLPSPRAWDVVLCISG
TLVSCENALVVAIIVGTPAFRAPMFLLVGSLAVADLLAGLGLVLHFAADFC
IGSPEMSLMLVGVLAMAFTASIGSLLAITVDRYLSLYNALTYYSETTVTRT
YVMLALVWVGALGLGLVPVLAWNCRDGLTTCGVVYPLSKNHLVVLAIAFFM
VFGIMLQLYAQICRIVCRHAQQIALQRHLLPASHYVATRKGIATLAVVLGA
FAACWLPFTVYCLLGDADSPRLYTYLTLLPATYNSMINPVIYAFRNQDVQK
VLWAICCCCSTSKIPFRSRSPSDV

1. A method for the provision of an appetite control agent which method comprises using one or more agonists and/or antagonists of the G protein coupled receptor GPR3 as test compounds in one or more appetite control test procedures, and selecting an active compound for use as an appetite control agent.

2. A method for the provision of an appetite control agent which method comprises (i) screening for agonists and/or antagonists of GPR3 and (ii) using one or more agonists and/or antagonists so identified as test compounds in one or more appetite control test procedures, and selecting an active compound for use as an appetite control agent.

3. The use of an agonist of GPR3 as identified according to claim 1 or claim 2, as an appetite control agent.

4. The use of an antagonist of GPR3 as identified according to claim 1 or claim 2, as an appetite control agent.

5. A method of appetite control which method comprises administering to an individual a pharmaceutically effective amount of an appetite control agent identified according to the method of claim 1 or claim 2.

6. An antisense oligonucleotide which is complementary to all or a part of the nucleotide sequence shown in Seq. ID1.

7. A dominant negative mutant of GPR3.

8. A dominant positive mutant of GPR3.

9. The use of a mutant as claimed in claim 7 or claim 8 in evaluating the role of GPR3 in the control of appetite.

10. A transgenic non-human animal in which the GPR3 gene has been deleted, inactivated or modified.

11. The use of a transgenic animal as claimed in claim 10 in evaluating the effects of test compounds in appetite control and obesity.

12. Diagnostic antibodies raised against a GPR3 polypeptide for use in the detection of physiological eating disorders.


SEQUENCE LISTING (3 sequences)

SEQ ID NO: 1 - DNA, 1102 bp, Homo sapiens
cccaggaagagacccctgtttagaggcctgggggcattggaggggacagcggtatcctgggaagagccccagggcatgaatgtggggataaggcattgggaccctatcaggtatcctgaggagagactcccaccacgtatcctgagaagcacctcaccccctccagaccccaactcccatcacccagcttggtcagcttctcacaaggcctttctcctgcaggtaccatgatgtggggtgcaggcagccctctggcctggctctcagctggctcaggcaacgtgaatgtaagcagcgtgggcccagcagaggggcccacaggtccagccgcaccactgccctcgcctaggcctgggatgtggtgctctgcatctcaggcaccctggtgtcctgcgagaatgcgctagtggtggccatcatcgtgggcactcctgccttccgtgcccccatgttcctgctggtgggcagcctggccgtggcagacctgctggcaggcctgggcctggtcctgcactttgctgctgtcttctgcatcggctcagcggagatgagcctggtgctggttggcgtgctggcaatggcctttaccgccagcatcggcagtctactggccatcactgtcgaccgctacctttctctgtacaatgccctcacctactattcagagacaacagtgacacggacctatgtgatgctggccttagtgtggggaggtgccctgggcctggggctgctgcctgtgctggcctggaactgcctggatggcctgaccacatgtggcgtggtttatccactctccaagaaccatctggtagttctggccattgccttcttcatggtgtttggcatcatgctgcagctctacgcccaaatctgccgcatcgtctgccgccatgcccagcagattgcccttcagcggcacctgctgcctgcctcccactatgtggccacccgcaagggcattgccacactggccgtggtgcttggagcctttgccgcctgctggttgcccttcactgtctactgcctgctgggtgatgcccactctccacctctctacacctatcttaccttgctccctgccacctacaactccatgatc

SEQ ID NO: 2 - protein, 330 aa, Homo sapiens
MMWGAGSPLAWLSAGSGNVNVSSVGPAEGPTGPAAPLPSPKAWDVVLCISGTLVSCENALVVAIIVGTPAFRAPMFLLVGSLAVADLLAGLGLVLHFAAVFCIGSAEMSLVLVGVLAMAFTASIGSLLAITVDRYLSLYNALTYYSETTVTRTYVMLALVWGGALGLGLLPVLAWNCLDGLTTCGVVYPLSKNHLVVLAIAFFMVFGIMLQLYAQICRIVCRHAQQIALQRHLLPASHYVATRKGIATLAVVLGAFAACWLPFTVYCLLGDAHSPPLYTYLTLLPATYNSMINPIIYAFRNQDVQKVLWAVCCCCSSSKIPFRSRSPSDV

SEQ ID NO: 3 - protein, 330 aa, Mus musculus
MMWGAGSSMAWFSAGSGSVNVSSVDPVEEPTGPATLLPSPRAWDVVLCISGTLVSCENALVVAIIVGTPAFRAPMFLLVGSLAVADLLAGLGLVLHFAADFCIGSPEMSLMLVGVLAMAFTASIGSLLAITVDRYLSLYNALTYYSETTVTRTYVMLALVWVGALGLGLVPVLAWNCRDGLTTCGVVYPLSKNHLVVLAIAFFMVFGIMLQLYAQICRIVCRHAQQIALQRHLLPASHYVATRKGIATLAVVLGAFAACWLPFTVYCLLGDADSPRLYTYLTLLPATYNSMINPVIYAFRNQDVQKVLWAICCCCSTSKIPFRSRSPSDV